(19) United States
(12) Patent Application Publication
(10) Pub. No.: US 2019/0269859 A1
(43) Pub. Date: Sep. 5, 2019

(54) DRUG INJECTION DEVICE WITH DEFLECTABLE HOUSING PORTION

(71) Applicant: Novo Nordisk A/S, Bagsvaerd (DK)

(72) Inventor: Nikolaj Eusebius Jacobsen, Soeborg (DK)

(21) Appl. No.: 16/343,470

(22) PCT Filed: Oct. 31, 2017
(86) PCT No.: PCT/EP2017/077850

§ 371 Date: Apr. 19, 2019

(30) Foreign Application Priority Data
Oct. 31, 2016 (EP) 16196602.3

(51) Int. Cl. A61M 5/315

(52) U.S. Cl. CPC A61M 5/31568; A61M 5/31551; A61M 2205/50; A61M 2005/3125; A61M 2205/583; A61M 2205/0294; A61M 2205/3375; A61M 2205/3327

(57) ABSTRACT

The present invention provides a drug injection device (1) comprising a housing (2) extending along a longitudinal axis and comprising an exterior housing surface, an activation element (20, 50) configured to undergo movement relative to the housing (2) corresponding to an action performed on or by the drug injection device (1), a transducer (90, 91, 92) arranged on a transducer bearing portion (8.1, 9.1, 9.2) of the exterior housing surface, a processor (82) electrically connected with the transducer (90, 91, 92), and an energy source (95). The transducer bearing portion (8.1, 9.1, 9.2) is capable of deflection relative to other portions of the exterior housing surface, the activation element (20, 50) is arranged to deflect the transducer bearing portion (8.1, 9.1, 9.2) in accordance with said movement relative to the housing (2), the transducer (90, 91, 92) is adapted to register a deflection of the transducer bearing portion (8.1, 9.1, 9.2), and the processor (82) is configured to count deflections registered by the transducer (90, 91, 92).

### FIELD OF THE INVENTION

The present invention relates generally to devices for delivering medicine to a subject, and more specifically to injection devices capable of setting and expelling one or more doses of drug from a drug reservoir.

### BACKGROUND OF THE INVENTION

In the diabetes care segment parenteral drug administration carried out using a traditional vial and syringe system is increasingly being substituted by administration using a pen injection device. Pen injection devices are particularly convenient in that they allow the user to perform a dosed injection from a prefilled drug reservoir without first having to manually transfer the particular dose from one reservoir (the vial) to another (the syringe).

Predominantly, two types of pen injection devices are available, durable injection devices being capable of delivering one or more doses of drug from a prefilled drug cartridge which can be loaded into the device before use and replaced after exhaustion, and disposable injection devices being capable of delivering one or more doses of drug from a prefilled and non-exchangeable drug cartridge. Each of these types of pen injection devices are, or may in principle be, realised in various sub-types, such as e.g. single shot devices adapted to deliver only one dose from a drug cartridge, multi-shot devices capable of delivering a plurality of doses from a drug cartridge, manual devices, where the user provides the force needed for injection, automatic devices having a built-in energy source releasable to occasion the injection, fixed dose devices adapted to deliver a predetermined dose of drug, variable dose devices offering delivery of different doses of drug, settable by the user, etc.

As the labels suggest a durable injection device is intended for use over a considerable period of time during which multiple drug cartridges are exhausted and replaced, whereas a disposable injection device is intended for use until its dedicated drug cartridge is exhausted, after which the entire injection device is discarded.

In the treatment of diabetes it is advisable to keep a log of the administered doses of a particular drug (e.g. insulin or glp-1), as well as the respective times of dose administration. Some injection devices accordingly offer electronic dose capturing and the opportunity to review dose related information on a digital display.

As an example, U.S. Pat. No. 6,277,099 B1 (Becton, Dickinson and Company) discloses an electronic medication delivery pen, wherein a dialled dose is detected by a piezoelectric sensor arrangement, activated in response to rotation of a user manipulable dose knob, and displayed on a liquid crystal display. The medication delivery pen also comprises a memory function, which together with the liquid crystal display provides an operable interface for conveying the dose size and the time of the last five injections.

US 2015/0302818 A1 (Owen Mumford Limited) discloses the use of an electronic paper display device in addition to a conventional scale drum merely to enable dose display of a larger font size. The electronic display is driven by signals from piezoelectric elements which are successively energised during rotation of a dose setting knob.

Until recently, the use of electronic features like the ones above has been limited to durable injection devices, as the additional cost connected with an inclusion of such features in a disposable injection device has been considered to lead to an economically unviable end product. However, the advances within particularly printed electronics are promising vis-à-vis the possibility of producing disposable injection devices with integrated electronic components at a reasonable cost.

WO 2015/071354 A1 (Novo Nordisk A/S) discloses a drug delivery device having a flexible sheet mounted at least in part to the exterior of its housing, the flexible sheet carrying e.g. printed electronic components such as a display, a processor, an energy source, and input means actuatable by a an action performed on or by the device. The display is configured to visually indicate e.g. the size of a set dose, the size of an expelled dose, and/or a time parameter in response to actuation of the input means. The input means are exemplified by various switch structures, each adapted to provide connection to an interior device component through an opening in the housing.

### SUMMARY OF THE INVENTION

It is an object of the invention to eliminate or reduce at least one drawback of the prior art, or to provide a useful alternative to prior art solutions.

In particular, it is an object of the invention to provide a drug injection device having means for enabling detection of a change of state of the drug injection device.

It is a further object of the invention to provide a drug injection device having means for electronic determination of a set and/or expelled dose of drug.

It is an even further object of the invention to provide such a drug injection device which is relatively simple and inexpensive to produce.

It is an even further object of the invention to provide a drug injection device having means for providing a real-time electronic visual indication of a state and/or a change of state of the drug injection device.

In the disclosure of the present invention, aspects and embodiments will be described which will address one or more of the above objects and/or which will address objects apparent from the following text.

A drug injection device embodying the principles of the present invention comprises a housing having a deflectable exterior housing surface portion capable of deflection relative to other exterior housing surface portions, and an activation element configured to undergo movement relative to the housing corresponding to an action performed on or by the injection device and to actuate, i.e. deflect, the deflectable exterior housing surface portion during said movement.

In such a drug injection device the deflectable exterior housing surface portion thus moves relative to other exterior housing surface portions when actuated. In the course of the action performed on or by the drug injection device the activation element may cause a single deflection or multiple deflections of the deflectable exterior housing surface portion. Each deflection may signify e.g. a change of state of the drug injection device, and the above arrangement accordingly enables detection of such change of state.

If the drug injection device further comprises a transducer arranged on the deflectable exterior housing surface portion for registering deflections thereof, a processor electrically connected with the transducer, and an energy source then any deflection of the deflectable exterior housing surface portion may be electronically detected.

If the drug injection device further comprises an electronic display electrically connected with the processor and adapted to convey information indicative of a state and/or a change of state of the drug injection device, and the processor is configured to update the electronic display corresponding to registered deflections of the deflectable exterior housing surface portion, then any information inferred from the detected deflection may be visually represented for the benefit of the user.

The state of the drug injection device may e.g. refer to a condition, wherein the drug injection device is prepared to expel a certain set dose of drug, or to a condition, wherein the drug injection device has expelled some or all of a dose of drug. The change of state of the drug injection device may e.g. refer to a set dose being changed, a dose delivery progression, or simply a mode switch such as a switching between a dose setting mode and a dose expelling mode. In accordance therewith an action performed on or by the injection device may e.g. be a dose setting action, a dose expelling action, or a mode switching action.

Hence, in a first aspect the invention provides a drug injection device according to claim 1.

Such a drug injection device provides for electronic detection of events like e.g. dose setting and/or dose expelling, thereby enabling electronic logging and presentation, potentially on another device, of treatment related data. The arrangement of the transducer on a deflectable portion of the exterior housing surface provides for an inexpensive and simple way of detecting details of an event without requiring electrical connection through the housing.

The movement of the activation element relative to the housing may be or comprise a rotational movement about the longitudinal axis, an axial movement along the longitudinal axis, or a transversal movement away from and/or towards to the longitudinal axis. For example, a mode switching action may be performed by urging the activation element axially with respect to the housing. During this axial movement the activation element may interface and deflect the transducer bearing portion axially relative to the other portions of the exterior housing surface.

In particular embodiments of the invention the movement of the activation element relative to the housing is a rotational movement about the longitudinal axis, and the deflection of the transducer bearing portion relative to the other portions of the exterior housing surface is a radial deflection. This provides for a very simple mechanical actuation mechanism, particularly useful in connection with dose setting actions and/or dose expelling actions, as will be clear from the below.

The transducer may be or comprise a piezoelectric sensor element, formed or mounted on the transducer bearing portion. For example, the transducer may comprise piezoelectric material printed on the transducer bearing portion or printed on a substrate covering the transducer bearing portion. Alternatively, the transducer may be or comprise e.g. a strain gauge, a galvanic foil sensor, or a capacitive sensor having an elastic dielectric between two electrodes.

The drug injection device may further comprise an electronic display electrically connected with the processor, and the processor may be configured to update the electronic display responsive to a deflection registered by the transducer. This allows for a direct visible reflection of a particular action being performed on or by the drug injection device.

The electronic display may be or comprise a flexible display, e.g. formed or mounted on a display carrying portion of the exterior housing surface. For example, the electronic display may be at least partially printed on the display carrying portion or on a substrate covering the display carrying portion. In particular, the electronic display may be an electrochromic display, e.g. having 14 or 16 individually controlled display segments, allowing e.g. a display of numbers from 0 to 99, or to 199. Alternatively, the electronic display may e.g. comprise a liquid crystal display panel mounted on the display carrying portion.

The transducer bearing portion and the display carrying portion may be spaced apart or partially overlapping. In particular embodiments of the invention the transducer bearing portion and the display carrying portion are spaced apart so as to allow the electronic display to be positioned on a non-deflectable portion of the exterior housing surface.

The processor and the energy source may also be formed or mounted on the exterior housing surface, thereby enabling the provision of a drug injection device, wherein all electronic components are arranged on the exterior of the housing and electrical connections through the housing are avoided. For example, the processor and/or the energy source, e.g. a battery, may be printed on respective exterior housing surface portions or printed on the same or respective substrates covering these respective exterior housing surface portions. Thus, all electronic components can be handled and kept outside the housing, while the assembly of components in the housing may involve handling of mechanical elements only.

The transducer, the processor, and optionally the electronic display and/or the energy source may be arranged on a single, flexible sheet which is mounted, at least in part, on the housing. The arrangement of the electronic components on a flexible sheet allows for faster and cheaper mass production in that multiple sheets may be prepared in a continuous process, e.g. by roll-to-roll printing.

In particular embodiments of the invention, the flexible sheet comprises a front and a back, where the transducer, the processor, and optionally the electronic display and/or the energy source are formed or mounted on the front, and where at least a portion of the back is adhered to the housing.

A flexible sheet carrying printed electronic components can be arranged in conformity with the exterior housing surface so as to add insignificantly to the outer dimensions of the housing, enabling the provision of a slender drug delivery device offering electronic dose capturing and, in some embodiments, having an electronic dose display. The various electronic components may naturally be electrically connected by respective conductors also printed on the flexible sheet.

As an alternative to being formed or mounted on the exterior housing surface the energy source, e.g. in the form of a button cell, may be arranged within the housing, or within a component mechanically coupled to the housing.

Further electronic components may be formed or mounted on the exterior housing surface or on the flexible sheet, such as e.g. an antenna for wirelessly relaying data to an external receiver.

The drug injection device inherently comprises a drug expelling mechanism for expelling a dose of drug from a reservoir. The reservoir may be pre-attached to the housing and prevented from being exchanged, or it may be attachable to the housing by the user, e.g. removably to allow for subsequent attachment of a replacement reservoir.

In specific embodiments of the invention the activation element forms part of the drug expelling mechanism and is adapted to rotate unidirectionally about the longitudinal axis during dose expelling in correspondence with the expelled dose. During this rotation the activation element deflects the transducer bearing portion a number of times which correlates with the number of units of medicament delivered. Each deflection of the transducer bearing portion is registered by the transducer and causes the transducer to emit a signal in response. The processor is configured to update the electronic display according to the signals received from the transducer. This way, a real-time display of a dose expelling progression is provided as a count-down from either a set dose or a predetermined fixed dose to a dose completed indication, such as e.g. a display of 0 or some dedicated acknowledgement.

If the reservoir is a variable volume reservoir comprising a movable wall, such as e.g. a cartridge comprising a slidable piston, the drug expelling mechanism may comprise a piston rod member, arranged in a guide structure in the housing (or a guide structure fixedly connected to the housing), for actuating the movable wall during a drug expelling action, and the activation element may be a piston rod follower, being rotationally locked to the piston rod, or even a piston rod driver, which when rotating about the longitudinal axis causes movement of the piston rod member through the guide structure. The piston rod follower or piston rod driver may comprise a first interface structure, such as e.g. a toothed rim, and an interior surface of the transducer bearing portion may comprise a second interface structure, such as e.g. a protrusion, enabling intermittent interaction with the first interface structure. An angular displacement of the piston rod follower, or piston rod driver, may thus be correlated with a resulting axial displacement of the piston rod member such that each deflection of the transducer bearing portion corresponds to a predefined volume, e.g. one unit, of drug expelled from the variable volume reservoir.

The piston rod member and the guide structure may be threadedly connected, while the piston rod member and the piston rod follower, or piston rod driver, are rotationally interlocked but capable of relative axial motion. In that case the guide structure may comprise a nut member, or at least a protrusion adapted to serve as a helical track follower. Alternatively, the piston rod member and the piston rod driver may be threadedly connected, while the piston rod member and the guide structure are rotationally interlocked but capable of relative axial motion. In that case the guide structure may comprise a spline.

The drug expelling mechanism may further comprise energy storage means, e.g. in the form of a spring member or a pressurised gas, releasable to cause a rotation of the activation element about the longitudinal axis.

The drug injection device may further comprise a dose setting mechanism operable to set a dose of drug to be expelled from the reservoir by the drug expelling mechanism.

In other specific embodiments of the invention, the activation element forms part of the dose setting mechanism and is adapted to rotate about the longitudinal axis during dose setting in correspondence with the selected dose. During this rotation the activation element deflects the transducer bearing portion a number of times which correlates with the dose selected by the user.

In such embodiments the activation element may be adapted to rotate unidirectionally about the longitudinal axis, the transducer may be a single transducer element, and the transducer bearing portion may be a single deflectable housing portion. Each deflection of the transducer bearing portion may be registered by the transducer and the transducer may be caused to emit a signal in response thereto. The processor may be configured to update the electronic display according to the signals received from the transducer. This way, a real-time display of a dose setting procedure is provided as a count-up from e.g. a zero dose indication.

Alternatively, the transducer may comprise a first transducer element and a second transducer element, and the transducer bearing portion may comprise a forward indicating deflectable housing portion and a backward indicating deflectable housing portion. The first transducer element may be arranged on the forward indicating deflectable housing portion, e.g. as described above, while the second transducer element may be arranged on the backward indicating deflectable housing portion, also e.g. as described above.

The activation element may be adapted to rotate bidirectionally about the longitudinal axis and arranged to deflect both the forward indicating deflectable housing portion and the backward indicating deflectable housing portion during rotation in either direction.

The first transducer element may register each deflection of the forward indicating deflectable housing portion and emit a first transducer signal in response thereto, and the sec- and transducer element may register each deflection of the backward indicating deflectable housing portion and emit a second transducer signal in response thereto.

The processor may be configured to a) receive each first transducer signal and each second transducer signal, b) acknowledge a unique signal pair as two successively received transducer signals consisting of one first transducer signal and one second transducer signal, and c) register an incremental dose change for every acknowledged unique signal pair. In case the drug injection device comprises an electronic display the processor may further be configured to d) update the electronic display according to a registered incremental dose change.

In the present context the term “unique signal pair” is used to imply that two successively received transducer signals consisting of one first transducer signal and one second transducer signal are uniquely tied together, such that two successive incremental dose change registrations require four individual transducer signals (two first transducer signals and two second transducer signals) and thus cannot occur based on only three individual transducer signals.

In connection with c) registering an incremental dose change may comprise registering an incremental dose increase if for a given unique signal pair the received first transducer signal precedes the received second transducer signal and registering an incremental dose decrease if for the given unique signal pair the received second transducer signal precedes the received first transducer signal. This provides for a dose setting mechanism having a real-time electronic indication of both dial up and dial down actions.

If the activation element forms part of the dose setting mechanism it may form part of, or be connected to, a user operable dose setting member, such as a dose setting knob. The dose setting member may be coupled with a part of the drug expelling mechanism and be adapted to position the part of the drug expelling mechanism relative to the housing by rotation about the longitudinal axis.

The drug injection device may further comprise an electronic memory unit adapted to store information pertaining to a drug expelling action, and/or to a dose setting action, such as e.g. the time of the drug expelling action and/or the size of the dose expelled from the reservoir in the course of the drug expelling action. The electronic memory unit may e.g. be formed, such as printed, or mounted on the flexible sheet.

Any deflectable portion of the exterior housing surface may e.g. be or comprise a straight or curved cantilever beam structure, moulded as part of the housing or formed by removal of surrounding housing material. Alternatively, any deflectable portion of the exterior housing surface may e.g. be realised by thinning of surrounding housing material or by removing parts of the surrounding housing material while maintaining a plurality of bridging structures to thereby provide a web-like configuration.

The housing may be generally circular cylindrical or conical. Alternatively, an axially extending information display area of the housing comprising the deflectable portion(s) of the exterior housing surface may have a larger radius of curvature than other areas of the housing, such as e.g. an opposing axially extending area of the housing. In exemplary embodiments of the invention, the axially extending information display area of the housing has a first radius of curvature, and at least one other, e.g. the opposing, axially extending area of the housing has a second radius of curvature, and the first radius of curvature is more than 3 times larger than the second radius of curvature. In particular, the first radius of curvature may be 4-10 times larger than the second radius of curvature.

In specific embodiments of the invention, the first radius of curvature is 6-7 times larger than the second radius of curvature.

Depending on the chosen substrate and manufacturing process for a flexible label carrying various electronic components, e.g. as printed electronics, it may be challenging to achieve a reliable attachment, e.g. adhesion, of the label to a housing having a cross-sectional configuration and dimension in the form and size of conventional pen-type injection devices. An injection device having a housing with different radii of curvature, as described above, allows for arrangement of the label in a less strained condition on a less curved surface, while still maintaining a relatively small cross-sectional dimension of the housing, which is attractive from a user perspective.

In a second aspect of the invention a drug injection device is provided comprising a housing extending along a longitudinal axis and comprising an exterior housing surface, an activation element configured to undergo movement relative to the housing corresponding to an action performed on or by the drug injection device, a transducer arranged on a transducer bearing portion of the exterior housing surface, an electronic display, a processor electrically connected with the electronic display and the transducer and configured to control the electronic display, and an energy source, wherein the transducer bearing portion is capable of deflection relative to other portions of the exterior housing surface, the activation element is arranged to deflect the transducer bearing portion in accordance with said movement relative to the housing, the transducer is adapted to register a deflection of the transducer bearing portion, and the processor is configured to update the electronic display responsive to the deflection registered by the transducer.

This provides for a direct visual feedback of any deflection of the transducer bearing portion, enabling the user to instantly check e.g. the level of a set dose, if a set dose is being increased or decreased, and/or whether a dose expelling event is actually progressing.

In a third aspect of the invention a method of producing a drug injection device is provided, the method comprising (I) providing i) a fully or partly assembled device comprising a housing having an exterior housing surface, where the exterior housing surface comprises a deflectable portion and a non-deflectable portion, the deflectable portion being capable of deflection relative to the non-deflectable portion, and an activation element arranged to under-go movement relative to the housing corresponding to an action performed on or by the drug injection device and to deflect the deflectable portion during said movement, and ii) a flexible sheet on which is formed or mounted a transducer configured to emit a signal in response to being deflected, a processor electrically connected to the transducer, and optionally an energy source, and (II) mounting the flexible sheet onto the housing, positioning the transducer on the deflectable portion.

In a fourth aspect of the invention a method of producing a drug injection device is provided, the method comprising (I) providing i) a fully or partly assembled device comprising a housing having an exterior housing surface, where the exterior housing surface comprises a deflectable portion and a non-deflectable portion, the deflectable portion being capable of deflection relative to the non-deflectable portion, and an activation element arranged to under-go movement relative to the housing corresponding to an action performed on or by the drug injection device and to deflect the deflectable portion during said movement, and ii) a flexible sheet on which is formed or mounted a transducer configured to emit a signal in response to being deflected, an electronic display adapted to display one or more dose indicating characters, such as e.g. a number, a processor electrically connected to the transducer and the electronic display and adapted to update the electronic display responsive to signals emitted by the transducer, and optionally an energy source, and (II) mounting the flexible sheet onto the housing, positioning the transducer on the deflectable portion.

Mounting of a transducer, such as a piezoelectric sensor, on a deflectable exterior housing surface for detection of deflections thereof provides for a simple and inexpensive construction of an event detecting drug injection device. The deflectable portion may be created easily, e.g. as part of the moulding process or by removing material from an adjacent exterior housing surface area to thereby form a cantilever arm, and the mounting of the transducer on the exterior of the housing obviates the need for mechanical and/or electrical access or connection to the housing interior.

Some manifestations of the invention are expressed in the following set of examples.

Example 1: A drug injection device comprising:
a housing extending along a longitudinal axis and comprising an exterior housing surface,
a drug expelling mechanism for expelling drug from a reservoir, the drug expelling mechanism comprising a first activation element configured to undergo a first movement relative to the housing corresponding to a drug expelling action,
a first transducer arranged on a first exterior housing surface portion, the first exterior housing surface portion being capable of a first deflection relative to other exterior housing surface portions, and the first activation element being arranged to cause first deflections in accordance with said first movement relative to the housing,
a dose setting mechanism for defining a dose of drug to be expelled from the reservoir by the drug expelling mechanism, the dose setting mechanism comprising a second activation element configured to undergo a second movement relative to the housing corresponding to a dose setting action,
a second transducer arranged on a second exterior housing surface portion, the second exterior housing surface portion being capable of a second deflection relative to other exterior housing surface portions, and the second activation element being arranged to cause second deflections in accordance with said second movement relative to the housing,
a third transducer arranged on a third exterior housing surface portion, the third exterior housing surface portion being capable of a third deflection relative to other exterior housing surface portions, and the second activation element being arranged to cause third deflections in accordance with said second movement relative to the housing,
a processor electrically connected with the first transducer, the second transducer, and the third transducer, and
an energy source,

wherein the first transducer is adapted to register first deflections and to emit respective first transducer signals in response thereto, the second transducer is adapted to register second deflections and to emit respective second transducer signals in response thereto, and the third transducer is adapted to register third deflections and to emit respective third transducer signals in response thereto, and

wherein the processor is configured to produce a first count of emitted first transducer signals and a second count of emitted seconds transducer signals and third transducer signals, where the second count is carried out by (a) incrementally increasing a current sum in response to receiving a first unique signal pair consisting of one second transducer signal and one third transducer signal in succession, where the second transducer signal precedes the third transducer signal, and (b) incrementally decreasing the current sum in response to receiving a second unique signal pair consisting of one second transducer signal and one third transducer signal in succession, where the third transducer signal precedes the second transducer signal.

This provides a drug injection device which is capable of electronic detection of both mechanical dose setting related motion and mechanical dose delivery related motion. The mechanical dose setting motion includes a dose increasing motion, where second deflections precede associated third deflections, and any dose decreasing motion, where third deflections precede associated second deflections. Notably, the drug injection device may be realised in an inexpensive and attractively small, e.g. slender, design.

Example 2: A drug injection device as in Example 1, wherein the first transducer, the second transducer, the third transducer, and the processor are arranged on a flexible sheet, and wherein the flexible sheet is mounted, at least in part, on the housing.

Example 3: A drug injection device as in Example 1 or 2, further comprising a memory unit, wherein the memory unit is configured to log an expelled dose of drug only when A) a dose setting action is followed by a drug expelling action, and B) the first count equals the second count.

Example 4: A drug injection device comprising:
a housing extending along a longitudinal axis and comprising an exterior housing surface,
a drug expelling mechanism for expelling drug from a reservoir, the drug expelling mechanism comprising a first activation element configured to undergo a first movement relative to the housing corresponding to a drug expelling action,
a first transducer arranged on a first exterior housing surface portion, the first exterior housing surface portion being capable of a first deflection relative to other exterior housing surface portions, and the first activation element being arranged to cause first deflections in accordance with said first movement relative to the housing,
a dose setting mechanism for defining a dose of drug to be expelled from the reservoir by the drug expelling mechanism, the dose setting mechanism comprising a second activation element configured to undergo a second movement relative to the housing corresponding to a dose setting action,
a second transducer arranged on a second exterior housing surface portion, the second exterior housing surface portion being capable of a second deflection relative to other exterior housing surface portions, and the second activation element being arranged to cause second deflections in accordance with said second movement relative to the housing,
a third transducer arranged on a third exterior housing surface portion, the third exterior housing surface portion being capable of a third deflection relative to other exterior housing surface portions, and the second activation element being arranged to cause third deflections in accordance with said second movement relative to the housing,
an electronic display capable of displaying one or more dose related characters,
a processor electrically connected with the electronic display, the first transducer, the second transducer, and the third transducer, the processor being configured to control the electronic display, and
an energy source,

wherein the first transducer is adapted to register first deflections and to emit respective first transducer signals in response thereto, the second transducer is adapted to register second deflections and to emit respective second transducer signals in response thereto, and the third transducer is adapted to register third deflections and to emit respective third transducer signals in response thereto, and

wherein the processor is configured to update the electronic display responsive to the respective first transducer signals, second transducer signals, and third transducer signals, the updating of the electronic display comprising (a) incrementally decreasing a current number on the electronic display in response to receiving a first transducer signal, (b) incrementally increasing the current number on the electronic display in response to receiving a first unique signal pair consisting of one second transducer signal and one third transducer signal in succession, where the second transducer signal precedes the third transducer signal, and (c) incrementally decreasing the current number on the electronic display in response to receiving a second unique signal pair consisting of one second transducer signal and one third transducer signal in succession, where the third transducer signal precedes the second transducer signal.

This provides a drug injection device which offers electronic detection and display of both mechanical dose setting related motion and mechanical dose delivery related motion in an inexpensive and attractively small, e.g. slender, design. The mechanical dose setting motion includes a dose increasing motion, where second deflections precede associated third deflections, and any dose decreasing motion, where third deflections precede associated second deflections. It is noted that a mechanical dose scale, such as e.g. a conventional scale drum, may be entirely omitted, reducing the number of mechanical components needed for the drug injection device.

Example 5: A drug injection device as in Example 4, wherein the electronic display, the first transducer, the second transducer, the third transducer, and the processor are arranged on a flexible sheet, and wherein the flexible sheet is mounted, at least in part, on the housing.

Example 6: A drug injection device as in Example 4 or 5, further comprising a memory unit, wherein the memory unit is configured to log an expelled dose of drug only when A) a dose setting action is followed by a drug expelling action, and B) the number of first transducer signals received during the drug expelling action equals the number of first unique signal pairs received during the dose setting action minus the number of second unique signal pairs received during the dose setting action.

Example 7: A drug injection device as in any of Example 2 or 5, wherein the energy source is arranged on the flexible sheet.

In relation to examples 3 and 6 a proper dose delivery may be registered by the processor when a sum of the first transducer signals received during a specific drug expelling action equals a sum of the first unique signal pairs received during a corresponding dose setting action minus a sum of the second unique signal pairs received during the corresponding dose setting action, i.e. when the first count equals the second count, because then the expelled dose corresponds to the set dose.

Accordingly, the electronic memory unit may be configured to log an expelled dose of drug only when the processor registers a proper dose delivery.

The drug injection device may further comprise a notification system configured to convey an alert to the user in response to the sum of the first transducer signals differing from the sum of the first unique signal pairs minus the sum of the second unique signal pairs. The alert may be visible, e.g. reflected on the electronic display, audible, e.g. produced by an additional loudspeaker, or tactile, e.g. produced by an included vibrator device.

As used herein the term “drug injection device” covers all types of devices for administering drug transcutaneously, i.e. including devices which are conventionally labelled injection devices (with or without an injection needle), where the drug is delivered over a relatively short time span, and devices which are conventionally labelled infusion devices, where the drug is delivered continuously over a longer period of time.

Also, as used herein, the terms “distal” and “proximal” denote positions at or directions along a drug delivery device, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is an exploded view of an injection device according to an exemplary embodiment of the invention,

FIG. 2 is a perspective view of the injection device of FIG. 1,

FIGS. 3a and 3b are longitudinal section views of a portion of the injection device showing, respectively, the injection device in a state just before setting of the first dose to be delivered and in a state just after delivery of the first dose, before release of the injection button,

FIGS. 4a and 4b are longitudinal section views of a portion of the injection device in another sectional plane,

FIG. 5 is a perspective view of a proximal portion of the injection device housing, showing the dose delivery detection mechanism,

FIG. 6 is a proximal perspective view of a proximal portion of the injection device housing, and

FIG. 7 is a cross-sectional view of the injection device, showing the dose setting detection mechanism.

In the figures like structures are mainly identified by like reference numerals.

### DESCRIPTION OF EXEMPLARY EMBODIMENTS

When in the following relative expressions, such as “clockwise” and “counter-clockwise”, “left” and “right”, etc. are used, these refer to the appended figures and not necessarily to an actual situation of use. The shown figures are schematic representations for which reason the configuration of the different structures as well as their relative dimensions are intended to serve illustrative purposes only.

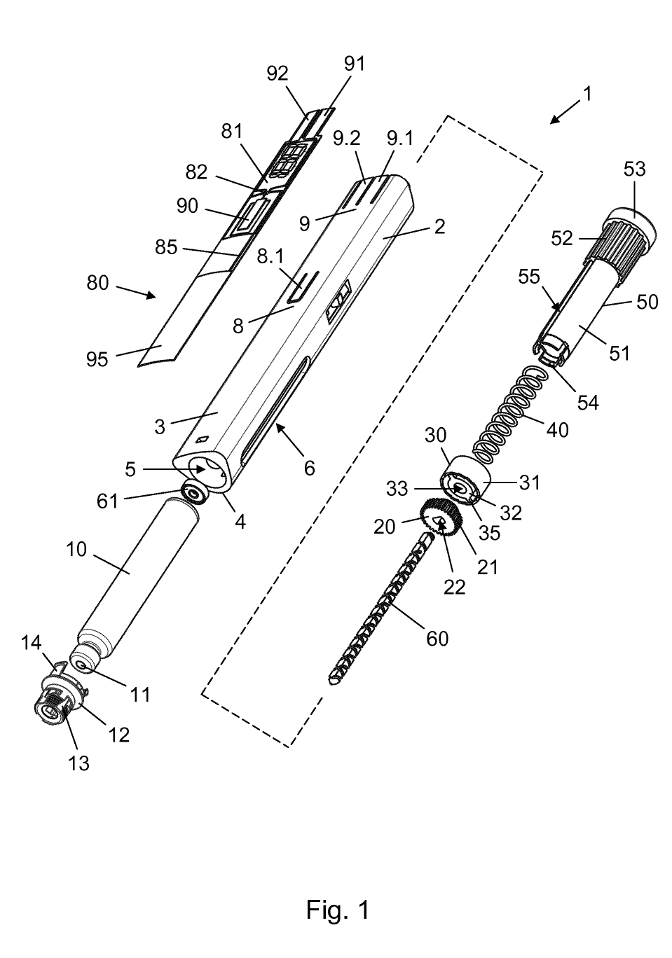

FIG. 1 is an exploded view of a pen injection device 1 according to an exemplary embodiment of the invention. The pen injection device 1 comprises a cylindrical housing 2 having a slightly curved information display surface 3 and a more conventionally curved opposing surface 4. The housing 2 accommodates a drug containing cartridge 10, which has been inserted through an opening 5 at a distal end thereof. The cartridge 10, which is closed at its distal end by a penetrable self-sealing septum 11 and at its proximal end by a slidable piston (not visible), is held within the housing 2 by a cartridge holder 12, being snapped to a proximal interior surface of the housing 2 by a pair of snap arms 14. The cartridge holder 12 further has a needle mount 13 and thereby serves as an attachment interface for an injection needle unit (not shown).

The housing 2 is provided with a longitudinal window 6 for inspection of the cartridge contents and further accommodates both a dose setting mechanism and an injection mechanism. The slidable piston in the cartridge 10 is adapted to be displaced by an elongated dual-threaded piston rod 60 of non-circular cross-section arranged to advance helically through a nut member 17 (see FIG. 3a) forming part of the housing 2. The piston rod 60 exerts a pressure on the slidable piston via a piston washer 61 as a result of being actuated by a setting nut 30, as will be described further below.

The setting nut 30 has an outer annular wall 31 and an inner nut structure 32 with a circular opening 33 for receiving the piston rod 60. The inner nut structure 32 is radially separated from the outer annular wall 31 by an opposing pair of spacer legs 35. A clutch 20 is arranged distally of the setting nut 30 and has a toothed rim 21 and a central opening 22 of non-circular configuration for mating engagement with the piston rod 60, providing a rotational interlocking of the two. The clutch 20 is axially movable within the housing 2 between a proximal dose setting position, in which it is rotationally locked to the housing 2, and a distal dose expelling position, in which it is free to rotate with respect to the housing 2.

A dose dial knob 50 extends into the housing 2 from a proximal end thereof. The dose dial knob 50 comprises a cylindrical main body 51 which is rotatable about a longitudinal axis of the housing 2 by manipulation of an end button 53. A corrugated collar 52 is provided at the main body 51 just distally of the end button 53. The end button 53 has a larger diameter than the main body 51 which serves to limit distal movement of the dose dial knob 50 relative to the housing 2. A number of hooked fingers 54 are provided at the distal end of the main body 51 for engagement with a hooked stub 23 (see FIG. 3a) on the clutch 20, providing an axially interlocking connection between the dose dial knob 50 and the clutch 20. A pair of opposing slots 55 extends longitudinally from the corrugated collar 52 to the hooked fingers 54. Each slot 55 is adapted to receive one of the spacer legs 35, providing a rotationally interlocked, but axially free, connection between the dose dial knob 50 and the setting nut 30.

A compression spring 40 is arranged to act between an interior surface of the end button 53 and a proximal surface of the setting nut 30.

With respect to the housing 2 in a central area 8 of the information display surface 3 some wall material has been removed to provide a radially deflectable central cantilever arm 8.1 and in a proximal area 9 of the information display surface 3 more wall material has been removed to provide a forward indicating radially deflectable proximal cantilever arm 9.1 and a backward indicating radially deflectable proximal cantilever arm 9.2.

A flexible label 80 is adhered to the information display surface 3. The label 80 carries printed electronics in the form of a display 81, a chip 82 comprising a processor and a memory module, a central piezo sensor 90, a first proximal piezo sensor 91, a second proximal piezo sensor 92, a battery 95, and various leads 85 electrically connecting the chip 82 with each of the other electronic components. The label 80 is mounted on the information display surface 3 such that the central piezo sensor 90 is positioned on the central cantilever arm 8.1, the first proximal piezo sensor 91 is positioned on the forward indicating radially deflectable proximal cantilever arm 9.1, and the second proximal piezo sensor 92 is positioned on the backward indicating radially deflectable proximal cantilever arm 9.2.

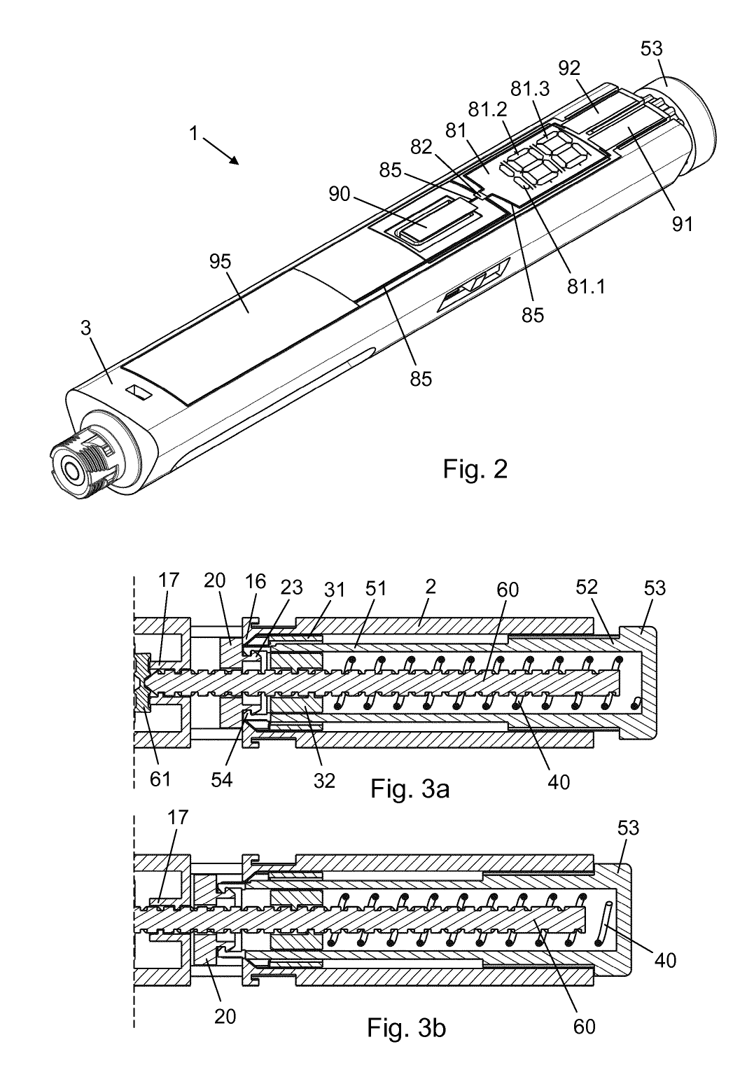

FIG. 2 shows the pen injection device 1 in an assembled state detailing the label 80 adhered to the information display surface 3. The display 81 is a 16-segment electrochromic display comprising a 2-segment hundreds digit 81.1 and two 7-segment tens, respectively units digits 81.2, 81.3. The display 81 is capable of showing dose numbers in the range [0 units; 199 units], controlled by the chip 82. The central piezo sensor 90 is bent during a radial deflection of the central cantilever arm 8.1 and will resultantly emit a short peak signal which is detected by the chip 82. Similarly, the first proximal piezo sensor 91 is bent during a radial deflection of the forward indicating radially deflectable proximal cantilever arm 9.1 and the second proximal piezo sensor 92 is bent during a radial deflection of the backward indicating radially deflectable proximal cantilever arm 9.2, each proximal piezo sensor 91, 92 emitting a signal to the chip 82 in response to being bent. The voltage output from any of the piezo sensors is sufficient to wake the processor in the chip 82.

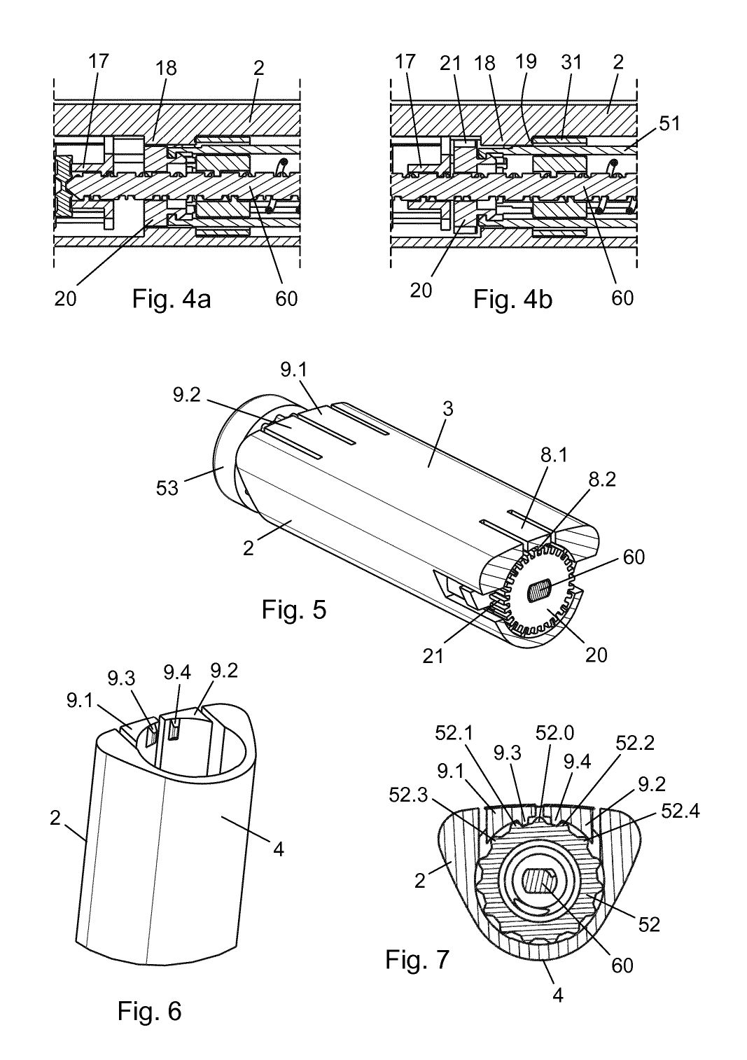

FIGS. 3a and 3b are both longitudinal section views of (approximately) the proximal half of the pen injection device 1, respectively just before setting of the first dose and just after completed expelling of the first dose. FIG. 4a is a longitudinal section view in another sectional plane of a part of the pen injection device 1 in the state shown in FIG. 3a, and FIG. 4b is, similarly, a longitudinal section view in another sectional plane of a part of the pen injection device 1 in the state shown in FIG. 3b.

Hence, FIGS. 3a and 4a show the interrelated positions of the various components in a dose setting state of the pen injection device 1. Specifically, it can be seen from FIG. 3a that in the dose setting state the end button 53 is axially spaced apart from the proximal end of the housing 2 by the compression spring 40, and that the interface between the hooked fingers 54 and the hooked stub 23 accordingly maintains the clutch 20 in the proximal dose setting position. In this position the clutch 20 is rotationally locked with respect to the housing 2 due to an engagement between the toothed rim 21 and a number of longitudinal splines 18 (see FIG. 4a) formed internally in the housing. The clutch 20 is prevented from further proximal displacement beyond this position by a stop surface 16 in the housing 2.

FIGS. 3b and 4b, on the other hand, show the interrelated positions of the various components in a drug expelling state of the pen injection device 1, more particularly at the end of a drug expelling action performed by the drug expelling mechanism, and while the end button 53 is depressed against the housing 2 (see FIG. 3b). It is noted that in such a depressed state of the dose dial knob 50 the hooked fingers 54 have forced the clutch 20 into the distal dose expelling position, where the toothed rim 21 is disengaged from the splines 18 (see FIG. 4b). It is also noted that at the end of a drug expelling action the outer annular wall 31 of the setting nut 30 rests against an end-of-dose stop 19 in the housing 2, constituted by respective proximal end surfaces of the splines 18 (see FIG. 4b).

FIG. 5 is a perspective view cross-sectioned to illustrate the interaction between the clutch 20 and the central cantilever arm 8.1 during a drug expelling action. When the clutch 20 rotates in the dose expelling position the toothed rim 21 moves along an interior wall surface of the housing 2 and as a tooth on the toothed rim 21 passes an inwardly directed protrusion 8.2 on the central cantilever arm 8.1 the central cantilever arm 8.1 is deflected radially outwardly and back, producing a click. The angular displacement of the clutch 20 is correlated with the amount of drug expelled, due to the rotational interlocked relationship between the clutch 20 and the piston rod 60, and in this embodiment each such returned deflection of the central cantilever arm 8.1 corresponds to one incremental unit of drug having been expelled.

FIG. 6 is a perspective view of the proximal end portion of the housing 2 showing interior wall portions of the proximal cantilever arms 9.1, 9.2. It can be seen that the forward indicating radially deflectable proximal cantilever arm 9.1 is provided with an inwardly directed protrusion 9.3 and the backward indicating radially deflectable proximal cantilever arm 9.2 is provided with another inwardly directed protrusion 9.4.

FIG. 7 is a cross-sectional view of the pen injection device 1 through a proximal end portion, illustrating the interaction between the corrugated collar 52 and the proximal cantilever arms 9.1, 9.2 during a dose setting action. As the corrugated collar 52 forms part of the dose dial knob 50 a rotation of the end button 53 causes a corresponding rotation of the corrugated collar 52. In the relative positions of the corrugated collar 52 and the housing 2 shown in FIG. 7 a central ridge 52.0 is positioned between the inwardly directed protrusions 9.3, 9.4, while a first left ridge 52.1 immediately to the left of the central ridge 52.0 is positioned adjacent to a left flank of the inwardly directed protrusion 9.3 and a first right ridge 52.2 immediately to the right of the central ridge 52.0 is positioned adjacent to a right flank of the inwardly directed protrusion 9.4.

A clockwise rotation of the end button 53 from this position will cause firstly the first left ridge 52.1 to start passing the inwardly directed protrusion 9.3, thereby deflecting the forward indicating radially deflectable proximal cantilever arm 9.1, and immediately thereafter the central ridge 52.0 to start passing the inwardly directed protrusion 9.4, thereby deflecting the backward indicating radially deflectable proximal cantilever arm 9.2.

Conversely, a counter-clockwise rotation of the end button 53 from the shown position will cause firstly the first right ridge 52.2 to start passing the inwardly directed protrusion 9.4, thereby deflecting the backward indicating radially deflectable proximal cantilever arm 9.2, and immediately thereafter the central ridge 52.0 to start passing the inwardly directed protrusion 9.3, thereby deflecting the forward indicating radially deflectable proximal cantilever arm 9.1. Each time a ridge on the corrugated collar 52 passes one of the inwardly directed protrusions 9.3, 9.4 a click is produced.

The angular displacement of the dose dial knob 50 is correlated with the axial displacement of the setting nut 30, due to the engagement between the respective spacer legs 35 and slots 55 and a threaded connection between the nut structure 32 and the piston rod 60, and thereby determines the dose set for delivery. In this embodiment each unique pair of deflections of the proximal cantilever arms 9.1, 9.2 caused by two neighbouring ridges on the corrugated collar 52 passing a respective one of the inwardly directed protrusions 9.3, 9.4 in succession corresponds to a change of the set dose by one incremental unit.

In other words, if for example the end button 53 is rotated clockwise from the position shown in FIG. 7 a change of the dose by one unit occurs when the first left ridge 52.1 has passed the inwardly directed protrusion 9.3 and the central ridge 52.0 has subsequently passed the inwardly directed protrusion 9.4, the angular displacement of these two ridges having produced a first forward indicating unique pair of deflections of the proximal cantilever arms 9.1, 9.2. Further clockwise rotation of the end button 53 will cause another change of the dose by one unit to occur when a second left ridge 52.3 immediately to the left of the first left ridge 52.1 has passed the inwardly directed protrusion 9.3 and the first left ridge 52.1 has subsequently passed the inwardly directed protrusion 9.4, the angular displacement of these two ridges having produced a second forward indicating unique pair of deflections of the proximal cantilever arms 9.1, 9.2, and so forth.

On the other hand, if the end button 53 is rotated counter-clockwise from the position shown in FIG. 7 a change of the dose by one unit occurs when the first right ridge 52.2 has passed the inwardly directed protrusion 9.4 and the central ridge 52.0 has subsequently passed the inwardly directed protrusion 9.3, the angular displacement of these two ridges having produced a first backward indicating unique pair of deflections of the proximal cantilever arms 9.1, 9.2. Further counter-clockwise rotation of the end button 53 will cause another change of the dose by one unit to occur when a second right ridge 52.4 immediately to the right of the first right ridge 52.2 has passed the inwardly directed protrusion 9.4 and the first right ridge 52.2 has subsequently passed the inwardly directed protrusion 9.3, the angular displacement of these two ridges having produced a second backward indicating unique pair of deflections of the proximal cantilever arms 9.1, 9.2, and so forth.

Specifically, each forward indicating unique pair of deflections causes an incremental increase of the set dose by one unit and each backward indicating unique pair of deflections causes an incremental decrease of the set dose by one unit.

In the following the invention will be described in connection with a use of the pen injection device 1 according to the present embodiment.

In FIG. 2 the pen injection device 1 is in the dose setting state, where the end button 53 is axially spaced apart from the housing 2. In this state a dose to be delivered from the cartridge 10 is set by the user rotating the end button 53 about the longitudinal axis.

The piston rod 60 comprises a first non-self-locking thread, which mates with a thread in the nut member 17 and an overlapping oppositely handed second non-self-locking thread which mates with a thread in the nut structure 32, providing a mechanical advantage of 2:1.

Due to the rotationally interlocked relationship between the setting nut 30 and the dose dial knob 50 the nut structure 32 will travel proximally along the second non-self-locking thread of the piston rod 60 when the end button 53 is rotated clockwise (seen from the proximal end of the pen injection device 1) in a dial up action, the clutch 20 in its dose setting position preventing the piston rod 60 from rotation relative to the housing 2. The proximal displacement of the setting nut 30 will compress the compression spring 40 which thereby stores energy.

The dose dial knob 50 is prevented from rotating in the absence of a user induced torque to the end button 53 due to the engagement between the corrugated collar 52 and the respective inwardly directed protrusions 9.3, 9.4, even when the compression spring 40 experiences a maximum in-use compression. An unintended distal return displacement of the setting nut 30 caused by a prematurely expanding compression spring 40 is thereby prevented.

Hence, as long as the clutch 20 is in its dose setting position, where the piston rod 60 is prevented from rotating, the setting nut 30 cannot perform a translational motion with respect to the housing 2 and can only perform a helical distal motion along the second non-self-locking thread, if the user rotates the end button 53 counter-clockwise in a dial down action. The compression spring 40 thus remains compressed when the user has completed the dose setting action.

In accordance with the setting nut 30 moving along the second non-self-locking thread of the piston rod 60 the ridges on the corrugated collar 52 passes the respective inwardly directed protrusions 9.3, 9.4, causing deflections of the forward indicating radially deflectable proximal cantilever arm 9.1 and the backward indicating radially deflectable proximal cantilever arm 9.2, as described above. At each deflection of the forward indicating radially deflectable proximal cantilever arm 9.1 the first proximal piezo sensor 91 emits a first sensor signal and at each deflection of the backward indicating radially deflectable proximal cantilever arm 9.2 the second proximal piezo sensor 92 emits a second sensor signal.

Each forward indicating unique pair of deflections in accordance with the above described thus prompts a forward indicating unique signal pair, SP_{f}, consisting of a first sensor signal followed by a second sensor signal. The chip 82 is configured to update the display 81 by an incremental increase in the form of a unit being added to the currently displayed number immediately upon registration of such a forward indicating unique signal pair.

Correspondingly, each backward indicating unique pair of deflections in accordance with the above described prompts a backward indicating unique signal pair, SP_{b}, consisting of a second sensor signal followed by a first sensor signal, and the chip 82 is configured to update the display 81 by an incremental decrease in the form of a unit being subtracted from the currently displayed number immediately upon registration of such a backward indicating unique signal pair. The display 81 accordingly provides a real-time electronic visual indication of the set dose.

A depression of the end button 53 against the housing 2 to expel a set dose (see FIG. 3b) causes the hooked fingers 54 to urge the clutch 20 distally into its dose expelling position, thereby disengaging the toothed rim 21 from the splines 18 (see FIG. 4b) and releasing the compression spring 40. The stored energy therefrom pushes the setting nut 30 distally, and the resulting translational motion of the nut structure 32 causes the piston rod 60 and the clutch 20 to rotate. The piston rod 60 is thus advanced helically through the nut member 17 which causes the piston washer 61 to advance the piston in the cartridge 10, thereby expelling the set dose of drug through an attached injection needle (not shown).

The drug expelling continues until an end surface of the outer annular wall 31 abuts the end-of-dose stop 19 at which point the piston rod 60, and thereby also the clutch 20, stops rotating. In accordance with the angular displacement of the clutch 20 during the drug expelling action the teeth of the toothed rim 21 pass the inwardly directed protrusion 8.2, causing deflections of the central cantilever arm 8.1, as described above. At each deflection of the central cantilever arm 8.1 the central piezo sensor emits a central sensor signal, S_{c}, and the chip 82 is configured to update the display 81 by an incremental decrease in the form of a unit being subtracted from the currently displayed number immediately upon registration of such a central sensor signal. The display 81 accordingly also provides a real-time electronic visual indication of the dose of drug being expelled.

During a normally progressing dose delivery the display 81 will show a dose count-down until the outer annular wall 31 reaches the end-of-dose stop 19 and the clutch 20 stops rotating, at which point a ‘0’ or some other indication of a completed drug expelling action will be displayed. However, the chip 82 is further configured to update the display 81 to display an error indication, such as e.g. ‘- -’, in case the number of received central sensor signals does not equal the number of registered forward indicating unique signal pairs minus the number of backward indicating unique signal pairs, i.e. in case ΣS_{c}≠ΣSP_{f}−SP_{b}.

For every normally progressing dose delivery (where ΣS_{c}=ΣSP_{f}−ΣSP_{b}) the chip 82 is configured to store a value representing the size of the dose delivered and a corresponding time of delivery. The stored data may be forwarded to, or requested by, an exterior device (not shown) via e.g. a wireless communication link (not shown).

1. A drug injection device comprising:
a housing extending along a longitudinal axis and comprising an exterior housing surface,
an activation element configured to undergo movement relative to the housing corresponding to an action performed on or by the drug injection device,
a transducer arranged on a transducer bearing portion of the exterior housing surface,
a processor electrically connected with the transducer, and
an energy source,
wherein
the transducer bearing portion is capable of deflection relative to other portions of the exterior housing surface,
the activation element is arranged to deflect the transducer bearing portion in accordance with said movement relative to the housing,
the transducer is adapted to register a deflection of the transducer bearing portion, and
the processor is configured to count deflections registered by the transducer.

2. A drug injection device according to claim 1, wherein the transducer and the processor are arranged on a flexible sheet, and wherein the flexible sheet is mounted, at least in part, on the housing.

3. A drug injection device according to claim 2, wherein the flexible sheet comprises a front and a back, wherein the transducer and the processor are formed or mounted on the front, and wherein at least a portion of the back is adhered to the housing.

4. A drug injection device according to claim 1, further comprising an electronic display electrically connected with the processor, wherein the processor is further configured to update the electronic display responsive to the deflection registered by the transducer.

5. A drug injection device according to claim 4, wherein the electronic display, the transducer, and the processor are arranged on a flexible sheet, and wherein the flexible sheet is mounted, at least in part, on the housing.

6. A drug injection device according to claim 5, wherein the flexible sheet comprises a front and a back, wherein the electronic display, the transducer, and the processor are formed or mounted on the front, and wherein at least a portion of the back is adhered to the housing.

7. A drug injection device according to claim 4, wherein the processor is electrically connected with the electronic display and the transducer by respective printed conductors.

8. A drug injection device according to claim 1, wherein the transducer comprises a printed piezoelectric material.

9. A drug injection device according to claim 1, wherein the movement of the activation element relative to the housing is a rotational movement about the longitudinal axis, and wherein the deflection of the transducer bearing portion is a radial deflection.

10. A drug injection device according to claim 1, further comprising a drug expelling mechanism for expelling a volume of drug from a reservoir, wherein the activation element forms part of the drug expelling mechanism and is adapted to rotate unidirectionally about the longitudinal axis during a drug expelling action in accordance with an expelled dose.

11. A drug injection device according to claim 10, wherein the activation element is configured to deflect the transducer bearing portion in response to undergoing a predetermined angular displacement relative to the housing, the predetermined angular displacement correlating with a unit of drug expelled from the reservoir.

12. A drug injection device according to claim 1, further comprising a dose setting mechanism for setting a dose of drug to be expelled, wherein the activation element forms part of the dose setting mechanism and is adapted to rotate about the longitudinal axis during a dose setting action in accordance with a selected dose.

13. A drug injection device according to claim 12, wherein the activation element is configured to deflect the transducer bearing portion in response to undergoing a predetermined angular displacement relative to the housing, the predetermined angular displacement correlating with an added dose unit.

14. A drug injection device according to claim 12, wherein the transducer bearing portion comprises a forward indicating deflectable housing portion and a backward indicating deflectable housing portion the transducer comprises a first transducer element arranged on the forward indicating deflectable housing portion and a second transducer element arranged on the backward indicating deflectable housing portion, and the activation element is adapted to rotate bidirectionally about the longitudinal axis and to deflect both the forward indicating deflectable housing portion and the backward indicating deflectable housing portion during rotation in either direction,
wherein the first transducer element registers each deflection of the forward indicating deflectable housing portion and emits a respective first transducer signal in response thereto, and the second transducer element registers each deflection of the backward indicating deflectable housing portion and emits a respective second transducer signal in response thereto, and
wherein the processor is configured to
receive each first transducer signal and each second transducer signal,
acknowledge a unique signal pair as two successively received transducer signals consisting of one first transducer signal and one second transducer signal, and
register an incremental dose increase for every acknowledged unique signal pair where the received first transducer signal precedes the received second transducer signal and an incremental dose decrease for every acknowledged unique signal pair where the received second transducer signal precedes the received first transducer signal.

15. A drug injection device according to claim 14, further comprising an electronic display electrically connected with the processor, wherein the processor is further configured to update the electronic display according to a registered incremental dose change.
